(19) United States
(12) Patent Application Publication
(10) Pub. No.: US 2011/0054341 A1
(43) Pub. Date: Mar. 3, 2011

(54) UNCONSTRAINED WEARABLE SPIROMETER APPARATUS, SYSTEM, AND MEASUREMENT METHOD USING THE SAME

(75) Inventors: Ji Wook Jeong, Daejeon (KR); Yong Won Jang, Daejeon (KR); Seung Hwan Kim, Daejeon (KR); Seon Hee Park, Daejeon (KR)

(73) Assignee: ELECTRONICS AND TELECOMMUNICATIONS RESEARCH INSTITUTE, Daejeon (KR)

(21) Appl. No.: 12/795,832

(22) Filed: Jun. 8, 2010

(30) Foreign Application Priority Data
Sep. 1, 2009 (KR) 10-2009-0082105

(51) Int. Cl. A61B 5/091

(52) U.S. Cl. 600/538

(57) ABSTRACT

Provided is an unconstrained wearable spirometer apparatus. The apparatus includes a variable resistor including a conductive yarn, the length of which changes according to a change in circumference of one's chest when breathing, and having a resistance which changes according to a change in length of the conductive yarn, a resistance-voltage converter converting the resistance of the variable resistor into a voltage, an analog-digital converter converting the voltage in an analog-digital manner, and digitizing the converted results, a cross-sectional capacity calculator calculating changes in cross-sectional capacity of one's chest according to the digitized voltage, an adder adding the calculated changes in cross-sectional capacity and calculating lung capacity, and a transmitter externally transmitting the calculated lung capacity information. Therefore, lung capacity can be measured by wearing such an apparatus in the form of clothing without the use of an external apparatus or device.

### CROSS-REFERENCE TO RELATED APPLICATION

This application claims priority to and the benefit of Korean Patent Application No. 10-2009-0082105, filed Sep. 1, 2009, the disclosure of which is incorporated herein by reference in its entirety.

### BACKGROUND

1. Field of the Invention

The present invention relates to a spirometer apparatus and a method of measuring lung capacity using the same, and more particularly, to an unconstrained wearable spirometer apparatus and a measurement method using the same.

2. Discussion of Related Art

When a respiration bio-signal such as lung capacity is measured, inhalation and exhalation are directly collected from outside the human body or inhalation and exhalation are filtered to electrically measure a rate of airflow.

However, such methods require external equipment whenever measurement is performed and the holding of an apparatus between the teeth.

### SUMMARY OF THE INVENTION

The present invention is directed to an unconstrained wearable spirometer apparatus that may be worn as clothing, detect a change in one's chest in real time, and estimate a change in lung volume, so that lung capacity can be measured.

One aspect of the present invention provides an unconstrained wearable spirometer apparatus including: a variable resistor including a conductive yarn, the length of which changes according to a change in circumference of one's chest when breathing, and having a resistance which changes according to a change in length of the conductive yarn; a resistance-voltage converter converting the resistance of the variable resistor into a voltage; an analog-digital converter converting the voltage in an analog-digital manner, and digitizing the converted results; a cross-sectional capacity calculator calculating changes in cross-sectional capacity of one's chest according to the digitized voltage; an adder adding the calculated changes in cross-sectional capacity and calculating lung capacity; and a transmitter externally transmitting the calculated lung capacity information.

The apparatus may further include a display module displaying lung capacity information calculated by the adder.

The variable resistor may include a plurality of conductive yarns spaced apart at uniform intervals.

The variable resistor may be in the form of a band or clothing.

The resistance-voltage converter may include a voltage divider circuit to convert the resistance of the variable resistor into a voltage.

The transmitter may externally transmit the lung capacity information using a wired or wireless communication method.

Another aspect of the present invention provides a method of measuring lung capacity using an unconstrained wearable spirometer apparatus including: measuring a resistance using a change in length of resistive conductive yarns according to a change in circumference of one's chest when breathing; converting the measured resistance into a voltage; converting the voltage in an analog-digital manner and digitizing the converted results; calculating changes in cross-sectional capacity of the chest according to the digitized voltage; adding the calculated changes in cross-sectional capacity and calculating lung capacity; and externally transmitting the calculated lung capacity information.

The method may further include displaying the lung capacity information after calculating the lung capacity.

The method may further include analyzing and displaying the externally transmitted lung capacity information after the calculated lung capacity information is externally transmitted.

The conductive yarns may be spaced apart at uniform intervals and be in the form of a band or clothing.

The voltage may be converted from the resistance, by a voltage divider circuit.

The lung capacity information may be externally transmitted using a wired or wireless communication method.

Still another aspect of the present invention provides to an unconstrained wearable spirometer system including: an unconstrained wearable spirometer apparatus including a variable resistor including a conductive yarn, the length of which changes according to a change in circumference of one's chest when breathing, and having a resistance which changes according to a change in length of the conductive yarn, and a reading module calculating lung capacity from the resistance of the variable resistor and externally transmitting the calculated lung capacity information; a signal analyzer analyzing the lung capacity information transmitted from the unconstrained wearable spirometer apparatus; and a signal display unit displaying the analyzed lung capacity information.

The reading module may include: a resistance-voltage converter converting the resistance of the variable resistor into a voltage; an analog-digital converter converting the voltage in an analog-digital manner and digitizing the converted results; a cross-sectional capacity calculator calculating changes in cross-sectional capacity of the chest according to the digitized voltage; an adder adding the calculated changes in cross-sectional capacity and calculating lung capacity; and a transmitter externally transmitting the calculated lung capacity information.

The variable resistor may include a plurality of conductive yarns spaced apart at uniform intervals, and may be in the form of a band or clothing.

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1A is a block diagram of a unconstrained wearable spirometer apparatus according to an exemplary embodiment of the present invention;

FIG. 1B illustrates a person wearing the unconstrained wearable spirometer apparatus of FIG. 1A;

FIG. 2 is a block diagram of a calculator illustrated in FIG. 1A;

FIG. 3 is a block diagram of an unconstrained wearable spirometer system according to an exemplary embodiment of the present invention;

FIG. 4 is a flowchart illustrating a method of measuring lung capacity using an unconstrained wearable spirometer apparatus according to an exemplary embodiment of the present invention; and

FIG. 5 is a graph showing changes in estimated lung capacity over time when a wearer breathes.

### DETAILED DESCRIPTION OF EMBODIMENTS

The present invention will be described more fully hereinafter with reference to the accompanying drawings, in which exemplary embodiments of the invention are shown. This invention may, however, be embodied in different forms and should not be construed as limited to the embodiments set forth herein. In the drawings, portions irrelevant to a description of the present invention are omitted for clarity, and like reference numerals denote like elements.

Throughout the specification, it will be understood that when a portion “includes” an element, it is not intended to exclude other elements but can further include other elements. Also, terms, such as “portion,” “system” and “module” may be used herein to refer to units for processing at least one function or operation which are implemented in hardware, software, or a suitable combination of both.

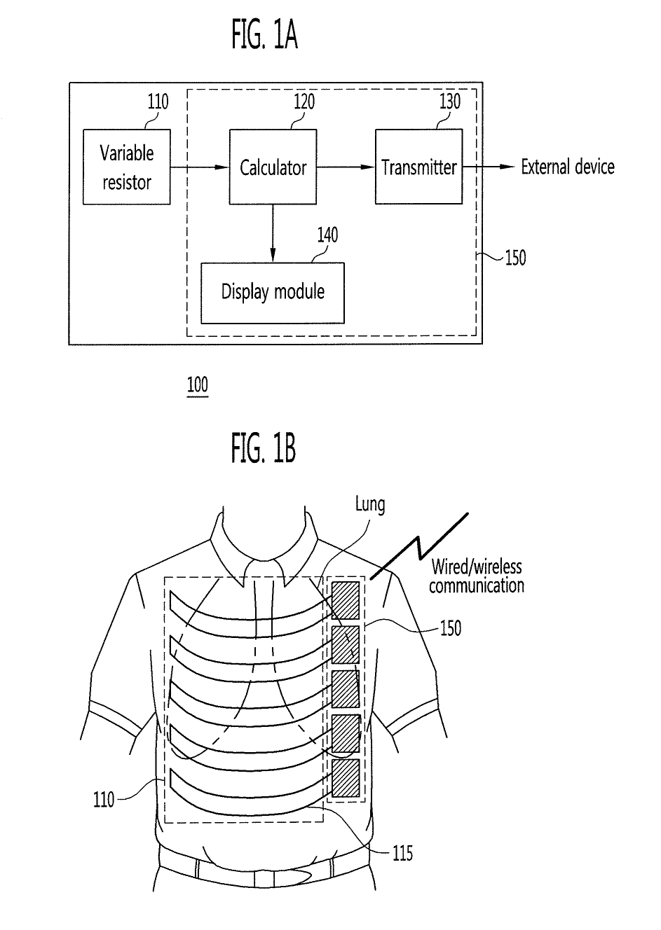

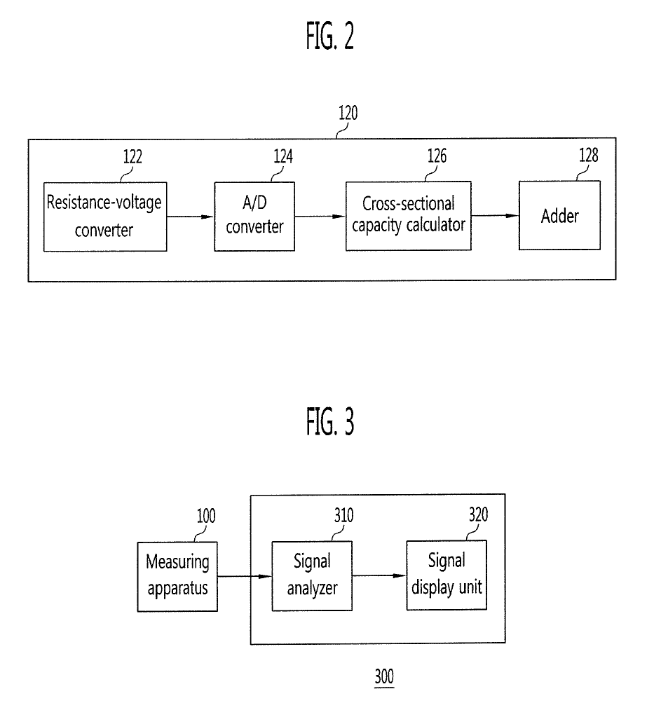

FIG. 1A is a block diagram of an unconstrained wearable spirometer apparatus according to an exemplary embodiment of the present invention, and FIG. 1B illustrates a person wearing the unconstrained wearable spirometer apparatus of FIG. 1A.

Referring to FIG. 1A, an unconstrained wearable spirometer apparatus 100 according to the present invention includes a variable resistor 110 and a reading module 150, and the reading module 150 includes a calculator 120, a transmitter 130 and a display module 140.

The variable resistor 110 includes a resistive conductive yarn, the length of which varies according to a change in circumference of one's chest in breathing. A resistance is changed according to a change in length of the conductive yarn. The variable resistor 110 includes a plurality of resistive conductive yarns, and the conductive yarns are spaced apart from each other on the chest at uniform intervals.

The conductive yarns include a conductive fiber and an elastic yarn. When elasticity of the elastic yarn causes the circumference of one's chest to be increased while breathing, the conductive yarns may be lengthened.

The conductive fiber includes a carbon fiber or a metal line. The conductive yarn may have a shape in which the conductive fiber is twisted with the elastic yarn. For example, the conductive fibers may be wound around the elastic yarn, or the conductive fibers may be wound twice in the opposite direction around the elastic yarn.

Referring to FIG. 1B, the variable resistor 110 including the plurality of resistive conductive yarns 115 spaced apart at uniform intervals may be formed in the shape of a band wrapping around a body to be independently worn. Alternatively, it may be inserted into clothing.

The calculator 120 performs conversion, changes and addition to calculate lung capacity from a resistance of the variable resistor 110. A detailed constitution of the calculator 120 will be described with reference to FIG. 2.

The transmitter 130 transmits lung capacity information calculated by the calculator 120 to an external device. The transmitter 130 may transmit the lung capacity information to the external device using a wired or wireless communication method.

The display module 140 displays the lung capacity information calculated by the calculator 120. It may inform users of results of measuring lung capacities through the display module 140 such as a liquid crystal display, etc.

FIG. 2 is a block diagram illustrating the calculator of FIG. 1A in more detail.

Referring to FIG. 2, the calculator 120 includes a resistance-voltage converter 122, an A/D converter 124, a cross-sectional capacity calculator 126 and an adder 128.

The resistance-voltage converter 122 converts a resistance of the variable resistor 110 into a voltage. For this purpose, the resistance-voltage converter 122 may include one or more voltage divider circuits.

The voltage divider circuit includes a reference resistance and a variable resistance that are serially connected between a power voltage terminal and a ground terminal. A portion formed of a resistive conductive yarn corresponds to the variable resistance. When breathing, the circumference of one's chest changes, causing the length of the resistive conductive yarn and the resistance to change, and the voltage applied to the variable resistance varies. Therefore, when the voltage is measured, the resistance may be converted into a voltage.

The A/D converter 124 converts an analog voltage converted by the resistance-voltage converter 122 into a digital voltage, and expresses the converted results numerically. The A/D converter 124 includes a plurality of A/D converting portions.

The cross-sectional capacity calculator 126 calculates a change in cross-sectional capacity of a chest according to the digital value digitized by the A/D converter 124. More specifically, a change in cross-sectional capacity is calculated by applying a conversion formula. For example, when a user wears a spirometer apparatus according to the present invention and inhales, the circumference of the user's chest increases, and thus the length of the conductive yarn also increases. Assuming that the user's upper body forms a cross-sectional sphere, the radius of the circle made by the user's body increases, and thus the area of the circle increases as well. Applying such a principle, a change in cross-sectional capacity of one's chest may be calculated.

The adder 128 adds the changes in cross-sectional capacity calculated by the cross-sectional capacity calculator 126 and calculates lung capacity. The conductive yarn of the variable resistor 110 may be repeatedly attached to a body within the height from shoulder to navel. Therefore, each change in cross-sectional capacity is added up per height, so that a change in volume of an effective lung capacity according to a breathing exercise may be calculated.

In the exemplary embodiment of the present invention, the variable resistor 110 includes a plurality of resistive conductive yarns, and each resistive conductive yarn may be connected to, e.g., a voltage divider circuit of the resistance-voltage converter 122, and to one of a plurality of A/D converting portions of the A/D converter. That is, a conductive yarn is connected to a voltage divider circuit, the cross-sectional capacity is calculated via one A/D converting portion, and the calculated cross-sectional capacity is repeatedly added with respect to the conductive yarns, so that lung capacity may be obtained.

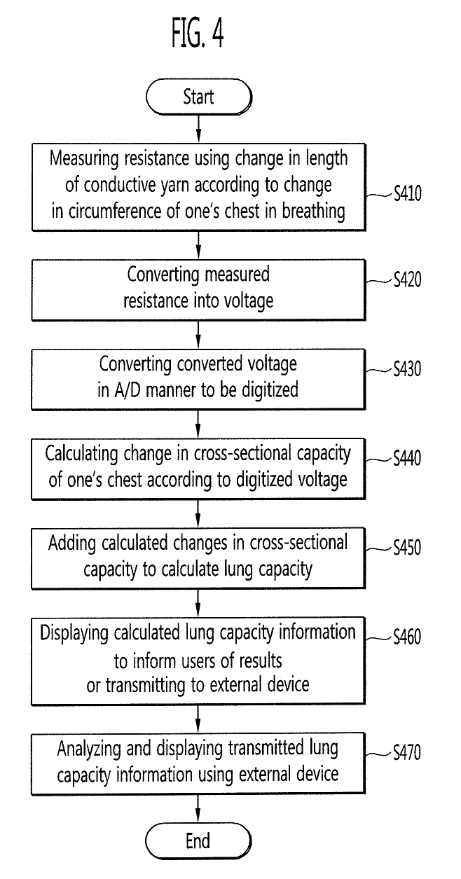

FIG. 3 is a block diagram of an unconstrained wearable spirometer system according to an exemplary embodiment of the present invention.

Referring to FIG. 3, the unconstrained wearable spirometer system of the present invention includes an unconstrained wearable spirometer apparatus 100, a signal analyzer 310, and a signal display unit 320. The signal analyzer 310 and the signal display unit 320 may be included in an external device 300 such as a personal digital assistant (PDA).

The signal analyzer 310 analyzes lung capacity information transmitted from the unconstrained wearable spirometer apparatus 100, and the signal display unit 320 displays the lung capacity information analyzed by the signal analyzer 310.

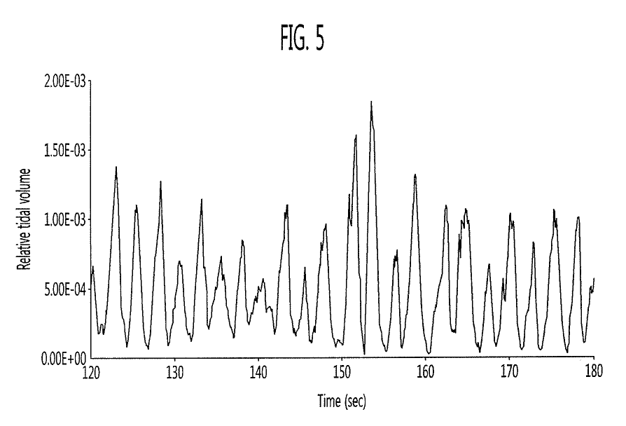

FIG. 4 is a flowchart illustrating a method of measuring lung capacity using an unconstrained wearable spirometer apparatus according to an exemplary embodiment of the present invention.

Since the detailed exemplary embodiment of a method of measuring lung capacity using the unconstrained wearable spirometer apparatus according to an exemplary embodiment of the present invention is as described above, the operation processes thereof will be briefly described.

Referring to FIG. 4, a resistance is measured using a change in length of a conductive yarn according to a change in circumference of one's chest in breathing (S410).

Afterwards, the measured resistance is converted into a voltage (S420), and the converted voltage is A/D converted to be digitized (S430).

Next, a change in cross-sectional capacity of one's chest according to the digitized voltage is calculated (S440), and the calculated changes in cross-sectional capacity are added to calculate lung capacity (S450).

Then, the calculated lung capacity information is displayed to inform users of the results or transmitted to an external device (S460).

Next, the transmitted lung capacity information is analyzed and displayed by the external device (S470).

FIG. 5 is a graph showing changes in estimated lung capacity over time when a wearer breathes.

Referring to FIG. 5, it is observed that when a wearer breathes, estimated lung capacity calculated based on a measured voltage changes in the same manner as a respiration cycle.

According to an unconstrained wearable spirometer apparatus according to the present invention, lung capacity can be measured by wearing such an apparatus in clothing form without the use of an external apparatus or device.

Further, such an apparatus enables serial monitoring, the results of measuring lung capacity to be displayed for users to see, and the results of measuring lung capacity to be transmitted to an external device using a wired/wireless communication method. Therefore, when any abnormal conditions (e.g., sleep apnea) arise during a breathing exercise of a user, such conditions can be noticed by others to prevent future accidents.

In the drawings and specification, there have been disclosed typical exemplary embodiments of the invention and, although specific terms are employed, they are used in a generic and descriptive sense only and not for purposes of limitation. As for the scope of the invention, it is to be set forth in the following claims. Therefore, it will be understood by those of ordinary skill in the art that various changes in form and details may be made therein without departing from the spirit and scope of the present invention as defined by the following claims.

What is claimed is:

1. An unconstrained wearable spirometer apparatus, comprising:
a variable resistor including a conductive yarn, the length of which changes according to a change in circumference of one's chest when breathing, and having a resistance which changes according to a change in length of the conductive yarn;
a resistance-voltage converter converting the resistance of the variable resistor into a voltage;
an analog-digital converter converting the voltage in an analog-digital manner, and digitizing the converted results;
a cross-sectional capacity calculator calculating changes in cross-sectional capacity of one's chest according to the digitized voltage;
an adder adding the calculated changes in cross-sectional capacity and calculating lung capacity; and
a transmitter externally transmitting the calculated lung capacity information.

2. The apparatus of claim 1, further comprising a display module displaying the lung capacity information calculated by the adder.

3. The apparatus of claim 1, wherein the variable resistor includes a plurality of conductive yarns spaced apart at uniform intervals.

4. The apparatus of claim 1, wherein the variable resistor is in the form of a band or clothing.

5. The apparatus of claim 1, wherein the resistance-voltage converter includes a voltage divider circuit in order to convert the resistance of the variable resistor into a voltage.

6. The apparatus of claim 1, wherein the transmitter externally transmits the lung capacity information using a wired or wireless communication method.

7. A method of measuring lung capacity using an unconstrained wearable spirometer apparatus, comprising:
measuring a resistance using a change in length of resistive conductive yarns according to a change in circumference of one's chest when breathing;
converting the measured resistance into a voltage;
converting the voltage in an analog-digital manner and digitizing the converted results;
calculating changes in cross-sectional capacity of the chest according to the digitized voltage;
adding the calculated changes in cross-sectional capacity and calculating lung capacity; and
externally transmitting the calculated lung capacity information.

8. The method of claim 7, further comprising displaying the lung capacity information after calculating the lung capacity.

9. The method of claim 7, further comprising analyzing and displaying the externally transmitted lung capacity information after the calculated lung capacity is externally transmitted.

10. The method of claim 7, wherein the conductive yarns are spaced apart at uniform intervals and in the form of a band or clothing.

11. The method of claim 7, wherein the voltage is converted from the resistance by a voltage divider circuit.

12. The method of claim 7, wherein the lung capacity information is externally transmitted using a wired or wireless communication method.

13. An unconstrained wearable spirometer system, comprising
an unconstrained wearable spirometer apparatus including a variable resistor including a conductive yarn, the length of which changes according to a change in circumference of one's chest when breathing, and having a resistance which changes according to a change in length of the conductive yarn, and a reading module calculating lung capacity from the resistance of the variable resistor and externally transmitting the calculated lung capacity information;
a signal analyzer analyzing the lung capacity information transmitted from the unconstrained wearable spirometer apparatus; and
a signal display unit displaying the analyzed lung capacity information.

14. The system of claim 13, wherein the reading module includes:
a resistance-voltage converter converting the resistance of the variable resistor into a voltage;
an analog-digital converter converting the voltage in an analog-digital manner and digitizing the converted results;
a cross-sectional capacity calculator calculating changes in cross-sectional capacity of the chest according to the digitized voltage;
an adder adding the calculated changes in cross-sectional capacity and calculating lung capacity; and
a transmitter externally transmitting the calculated lung capacity information.

15. The system of claim 14, wherein the variable resistor includes a plurality of conductive yarns spaced apart at uniform intervals, and is in the form of a band or clothing.
